(19) United States
(12) Patent Application Publication
(10) Pub. No.: US 2026/0248375 A1
(43) Pub. Date: Aug. 27, 2026

(54) LARYNGOSCOPE

(71) Applicant: SAFE Scope Inc., Etobicoke (CA)

(72) Inventor: Neil Dilworth, Toronto (CA)

(21) Appl. No.: 19/648,970

(22) Filed: Apr. 15, 2026

Related U.S. Application Data
(63) Continuation of application No. PCT/CA2024/051448, filed on Nov. 1, 2024.
(60) Provisional application No. 63/546,791, filed on Nov. 1, 2023.

(51) Int. Cl. A61B 1/267; A61B 1/00

(52) U.S. Cl. CPC A61B 1/267; A61B 1/00068; A61B 1/00119; A61B 1/00128; A61B 1/00135

(57) ABSTRACT

The present invention relates to a laryngoscope that can be used to intubate a patient while simultaneously providing oxygenation and suction at the level of the distal pharynx/proximal larynx to improve both oxygen availability and visualization of the patient's airway access during the intubation procedure. The laryngoscope comprises two lumens coupled to the handle and blade of the device, the lumens configured to connect directly to external sources, such as suction or oxygen. The present device permits the user to complete the intubation procedure without having to remove the device from the patient's airway before the intubation procedure is complete or have a second person insert and operate additional devices to supply oxygen and/or suction during the intubation procedure. A cover for a laryngoscope with the same features is also described. The present laryngoscope and cover are believed to have application in an emergency medical setting.

### CROSS-REFERENCE TO RELATED APPLICATION

The present application claims the benefit under 35 U.S.C. 119(e) of provisional patent application Ser. No. 63/546,791 filed Nov. 1, 2023, the contents of which are hereby incorporated by reference.

### BACKGROUND OF THE INVENTION

### Field of the Invention

In one of its aspects, the present invention relates to medical instruments, and more particularly to a laryngoscope configured to provide multiple functions—e.g., incorporating suction and oxygen flow.

### Description of the Prior Art

Emergency intubation, in which an endotracheal tube is inserted into a patient's trachea to provide airway patency, can be one of the most difficult and life-threatening procedures in a hospital emergency department. Indications for emergency intubation include trauma, anaphylaxis, angioedema, intracranial pathology, intoxication, respiratory distress, and upper gastrointestinal bleeding, and the like.

Orointubation, which is the most common route of intubation, requires a practitioner to guide an endotracheal tube through the patient's mouth, between the vocal cords, and into the trachea. Once the endotracheal tube is correctly inserted into the trachea, an open airway can be maintained and aspiration of gastric contents and bodily fluids can be prevented. In an emergency setting, intubation must be done quickly and accurately, failing which there is risk of possible injury or even death.

There are many factors associated with challenging intubation cases that can affect the accurate placement of the endotracheal tube. There are patient factors, including the patient's anatomy (e.g., cervical spine precautions/mobility, jaw opening mobility, altered/irregular pharyngeal and/or laryngeal anatomies, and the like) and physiological factors (e.g., cardiovascular, respiratory, metabolic, and the like), and environmental factors, including patient positioning and lighting, and the like.

Laryngoscopes are often used to assist in the insertion of endotracheal tubes, which assist in the visualization of the vocal cords to allow for correct placement of the endotracheal tube. Generally, laryngoscopes consist of a handle and a blade. The blades come in different sizes and may be straight or curved. Laryngoscopes may be used in direct laryngoscopy, in which the practitioner directly views the larynx, or fiberoptic laryngoscopy, in which a camera is incorporated into the use of the laryngoscope, allowing the larynx to be visualized indirectly by the practitioner on a screen.

While laryngoscopes can increase the speed and accuracy of the placement of the endotracheal tube, there remain unresolved challenges.

Gastric contents, bodily fluids, and foreign materials in the airway can obscure the practitioner's view and complicate intubation. Such substances can also obstruct the endotracheal tube. Maintaining adequate oxygenation of the patient during intubation can also be a challenge. Intubation patients are often pre-medicated for sedation and paralysis to facilitate insertion of the endotracheal tube and subsequent ventilation; however, as a result, the ability of these patients to breath on their own may be compromised. While patients are often pre-oxygenated prior to intubation, once the patient is pre-medicated and the laryngoscope is introduced into the airway, the patient does not receive oxygen until either the intubation is successful or the laryngoscope is removed and mechanical ventilation is applied. As a result, intubation must be done quickly and, preferably, successful on the first-pass, or there is a risk of brain damage or death.

Accordingly, despite the advances made to date in the development of laryngoscopes, there is room for improvement to address the above-mentioned problems and shortcomings of the prior art.

### SUMMARY OF THE INVENTION

It is an object of the present invention to obviate or mitigate at least one of the above-mentioned disadvantages of the prior art.

It is another object of the present invention to provide a novel laryngoscope device.

Accordingly in one of its aspects, the present invention provides a laryngoscope device of unitary construction comprising: a handle element at a proximal portion of the device; a blade element at a distal portion of the device, the blade element configured for insertion into an airway of a patient; and a first lumen and a second lumen coupled to at least a portion of the handle element or the blade element.

In another of its aspects, the present invention provides a laryngoscope device comprising: a handle element at a proximal portion of the device; a blade element at a distal portion of the device, the blade element configured for insertion into an airway of a patient; and a first lumen and a second lumen secured to at least a portion of the handle element or the blade element.

In another of its aspects, the present invention provides a cover for a laryngoscope, the laryngoscope comprising a handle element at a proximal portion of the laryngoscope and a blade element at a distal portion of the laryngoscope, the blade element being configured for insertion into an airway of a patient, the cover comprising: a sleeve comprising an open end and a closed end, the open end configured to reversibly receive the blade element and handle element of the laryngoscope, wherein the sleeve is configured to cover the blade element and at least a portion of the handle element; and a first lumen and a second lumen coupled to the sleeve.

Thus, the present inventor has developed a laryngoscope device or cover for a laryngoscope that incorporates dual lumens integrated in an optimized design for intubation and handling. The present device provides a means for intubating a patient while simultaneously providing oxygenation to the patient and suction to improve visualization of the patient's airway access during the intubation procedure. This permits the user to complete the intubation procedure without having to remove the laryngoscope from the patient's airway before the intubation procedure is complete, as may be the case if the patient requires oxygenation and/or if fluids are obscuring the user's view. It also eliminates the need for a second person to insert and operate additional devices to supply oxygen and/or suction during the intubation procedure, which can take up the limited space in the airway available for intubation. Using the present device, the user can visualize the correct placement of the endotracheal tube, while simultaneously providing suction and oxygenation using just one hand. The device is designed to connect directly to external sources of suction and oxygen, thereby reducing the set-up time needed for the intubation procedure and the equipment required. The dual lumen design may also add additional strength to the distal end of the device, thereby preventing breakage during use. With reduced set-up time, optimized design for single-handed use, improved strength, and less equipment required, the present device is believed to have application in an emergency medical setting.

To the knowledge of the inventor, a laryngoscope device having such a combination of features are heretofore unknown.

Other advantages of the invention will become apparent to those of skill in the art upon reviewing the present specification.

### BRIEF DESCRIPTION OF THE DRAWINGS

Embodiments of the present invention will be described with reference to the accompanying drawings, wherein like reference numerals denote like parts, and in which:

FIG. 1 is a right side perspective view of one embodiment of the present laryngoscope device.

FIG. 2 is a left side perspective view of the embodiment of the present laryngoscope device illustrated in FIG. 1.

FIG. 3 is a front view of the embodiment of the present laryngoscope device illustrated in FIG. 1.

FIG. 4 is a partial enlarged perspective view of the embodiment of the present laryngoscope device illustrated in FIG. 1.

FIG. 5 is a top view of the embodiment of the embodiment of the present laryngoscope device illustrated in FIG. 1.

FIG. 6 is a bottom view of the embodiment of the embodiment of the present laryngoscope device illustrated in FIG. 1.

FIG. 7(a) is a partial enlarged perspective view of the proximal portion of the present laryngoscope device illustrated in FIG. 1 and (b) a partial enlarged perspective view of the distal portion of the present laryngoscope device illustrated in FIG. 1.

FIG. 8 is a right side view of one embodiment of the present laryngoscope cover.

FIG. 9 is a left side view of the embodiment of the present laryngoscope cover illustrated in FIG. 8.

FIG. 10 is a front view of the embodiment of the present laryngoscope cover illustrated in FIG. 8.

FIG. 11 is a partial back view of the embodiment of the present laryngoscope cover illustrated in FIG. 8.

### DETAILED DESCRIPTION OF THE PREFERRED EMBODIMENTS

The present invention relates to a laryngoscope device of unitary construction comprising: a handle element at a proximal portion of the device; a blade element at a distal portion of the device, the blade element configured for insertion into an airway of a patient; and a first lumen and a second lumen coupled to at least a portion of the handle element or the blade element.

Preferred embodiments of this device may include any one of or a combination of any two or more of any of the following features:
the first and second lumen are coupled to a portion of both the handle element and the blade element;
the first and second lumen are coupled to the entirety of the handle element;
the first and second lumen are coupled to the entirety of the blade element;
the first and second lumen are coupled to the entirety of the handle element and blade element;
the first lumen and second lumen are disposed on a lateral side of the device;
the first lumen and second lumen are disposed on the same lateral side of the device;
the first and second lumen are disposed in a housing with a solid cross-section;
each lumen comprises a first end adjacent to a portion of the handle element and a second end adjacent to a portion of the blade element;
the first end of the first and second lumen extend beyond a proximal end of the handle element;
a distal portion of the blade extends beyond the second end of the first and second lumen;
the distal portion of the blade is configured to a tapered tip;
the first end of each lumen comprises a connector element;
the connector element of the first lumen is configured to reversibly connect the first lumen to a compressed gas source;
the compressed gas is oxygen;
the connector element of the second lumen is configured to reversibly connect the second lumen to a suction source;
the suction source is a vacuum pump;
the second end of each lumen comprises a port element;
the port element comprises a filter element;
one or both lumens comprise a valve element interposed between the lumen's first end and second end, the valve element configured to be reversibly movable from a first position to a second position, wherein when the valve element is in the first position, the first end and second end of the lumen are in fluid communication and when the valve element is in the second position, the first end and second end of the lumen are not in fluid communication;
the value element comprises an aperture in the lumen wall;
the device further comprises an activation means configured to selectively move the valve element between the first position and second position;
the device comprises a third lumen coupled to at least one of a portion of the handle element and the blade element;
the third lumen is coupled to a portion of both the handle element and the blade element;
the third lumen is coupled to the entirety of the handle element;
the third lumen is coupled to the entirety of the blade element;
third lumen is coupled to the entirety of the handle element and blade element;
the third lumen is disposed on a lateral side of the device;
the third lumen is disposed on the same lateral side of the device as the first and second lumen;
the third lumen is disposed in a housing with a solid cross-section;
the third lumen comprises a first end adjacent to a portion of the handle element and a second end adjacent to a portion of the blade element;
the first end of the third lumen comprises a connector element;
the connector element of the third lumen is configured to reversibly connect the third lumen to a source of medication;
the second end of the third lumen comprises a port element;
the port of the third lumen comprises a filter element;
the device comprises a chamber contained within and traversing through the handle element and blade element, the chamber being configured to reversibly receive and house an image capturing device;
one or both of the first and second lumen comprises a second port element proximal to its second end;
the device comprises a metal alloy, titanium, stainless steel, or plastic;
the blade element is curved; and
the device is disposable.

The present invention also relates to a laryngoscope device comprising: a handle element at a proximal portion of the device; a blade element at a distal portion of the device, the blade element configured for insertion into an airway of a patient; and a first lumen and a second lumen secured to at least a portion of the handle element or the blade element.

Preferred embodiments of this device may include any one of or a combination of any two or more of any of the following features:
the first and second lumen are secured to a portion of both the handle element and the blade element;
the first and second lumen are secured to the entirety of the handle element;
the first and second lumen are secured to the entirety of the blade element;
the first and second lumen are secured to the entirety of the handle element and blade element;
the first lumen and second lumen are disposed on a lateral side of the device;
the first lumen and second lumen are disposed on the same lateral side of the device;
the first and second lumen are disposed in a housing with a solid cross-section;
each lumen comprises a first end adjacent to a portion of the handle element and a second end adjacent to a portion of the blade element;
the first end of the first and second lumen extend beyond a proximal end of the handle element;
a distal portion of the blade extends beyond the second end of the first and second lumen;
the distal portion of the blade is configured to a tapered tip;
the first end of each lumen comprises a connector element;
the connector element of the first lumen is configured to reversibly connect the first lumen to a compressed gas source;
the compressed gas is oxygen;
the connector element of the second lumen is configured to reversibly connect the second lumen to a suction source;
the suction source is a vacuum pump;
the second end of each lumen comprises a port element;
the port element comprises a filter element;
one or both lumens comprise a valve element interposed between the lumen's first end and second end, the valve element configured to be reversibly movable from a first position to a second position, wherein when the valve element is in the first position, the first end and second end of the lumen are in fluid communication and when the valve element is in the second position, the first end and second end of the lumen are not in fluid communication;
the value element comprises an aperture in the lumen wall;
the device further comprises an activation means configured to selectively move the valve element between the first position and second position;
the device comprises a third lumen coupled to at least one of a portion of the handle element and the blade element;
the third lumen is coupled to a portion of both the handle element and the blade element;
the third lumen is coupled to the entirety of the handle element;
the third lumen is coupled to the entirety of the blade element;
third lumen is coupled to the entirety of the handle element and blade element;
the third lumen is disposed on a lateral side of the device;
the third lumen is disposed on the same lateral side of the device as the first and second lumen;
the third lumen is disposed in a housing with a solid cross-section;
the third lumen comprises a first end adjacent to a portion of the handle element and a second end adjacent to a portion of the blade element;
the first end of the third lumen comprises a connector element;
the connector element of the third lumen is configured to reversibly connect the third lumen to a source of medication;
the second end of the third lumen comprises a port element;
the port of the third lumen comprises a filter element;
the device comprises a chamber contained within and traversing through the handle element and blade element, the chamber being configured to reversibly receive and house an image capturing device;
one or both of the first and second lumen comprises a second port element proximal to its second end;
the device comprises a metal alloy, titanium, stainless steel, or plastic;
the blade element is curved; and
the device is disposable.

The present invention also relates to a cover for a laryngoscope, the laryngoscope comprising a handle element at a proximal portion of the laryngoscope and a blade element at a distal portion of the laryngoscope, the blade element being configured for insertion into an airway of a patient, the cover comprising: a sleeve comprising an open end and a closed end, the open end configured to reversibly receive the blade element and handle element of the laryngoscope, wherein the sleeve is configured to cover the blade element and at least a portion of the handle element; and a first lumen and a second lumen coupled to the sleeve.

Preferred embodiments of this cover may include any one of or a combination of any two or more of any of the following features:
the first lumen and second lumen are disposed on a lateral side of the cover;
the first lumen and second lumen are disposed on the same lateral side of the cover;
the first and second lumen are disposed in a housing with a solid cross-section;
each lumen comprises a first end adjacent to the open end of the sleeve and a second end adjacent to the closed end of the sleeve;
the first end of each lumen comprises a connector element;
the connector element of the first lumen is configured to reversibly connect the first lumen to a compressed gas source;
the compressed gas is oxygen;
the connector element of the second lumen is configured to reversibly connect the second lumen to a suction source;
the suction source is a vacuum pump;
the second end of each lumen comprises a port element;
the port element comprises a filter element;
one or both lumens comprise a valve element interposed between the lumen's first end and second end, the valve element configured to be reversibly movable from a first position to a second position, wherein when the valve element is in the first position, the first end and second end of the lumen are in fluid communication and when the valve element is in the second position, the first end and second end of the lumen are not in fluid communication;
the value element comprises an aperture in the lumen wall;
the cover further comprises an activation means configured to selectively move the valve element between the first position and second position;
the cover comprises a third lumen coupled to the sleeve;
the third lumen is disposed on a lateral side of the cover;
the third lumen is disposed on the same lateral side of the cover as the first and second lumen;
the third lumen is disposed in a housing with a solid cross-section;
the third lumen comprises a first end adjacent to the open end of the sleeve and a second end adjacent to the closed end of the sleeve;
the first end of the third lumen comprises a connector element;
the connector element of the third lumen is configured to reversibly connect the third lumen to a source of medication;
the second end of the third lumen comprises a port element;
the port of the third lumen comprises a filter element;
the cover comprises a substantially transparent material;
the cover comprises a flexible material;
the cover comprises a rigid material;
the cover comprises a polymeric material
the polymeric material is plastic; and
the cover is re-useable.

Preferred embodiments of the present invention will be described with reference to the following exemplary information which should not be used to limit or construe the invention.

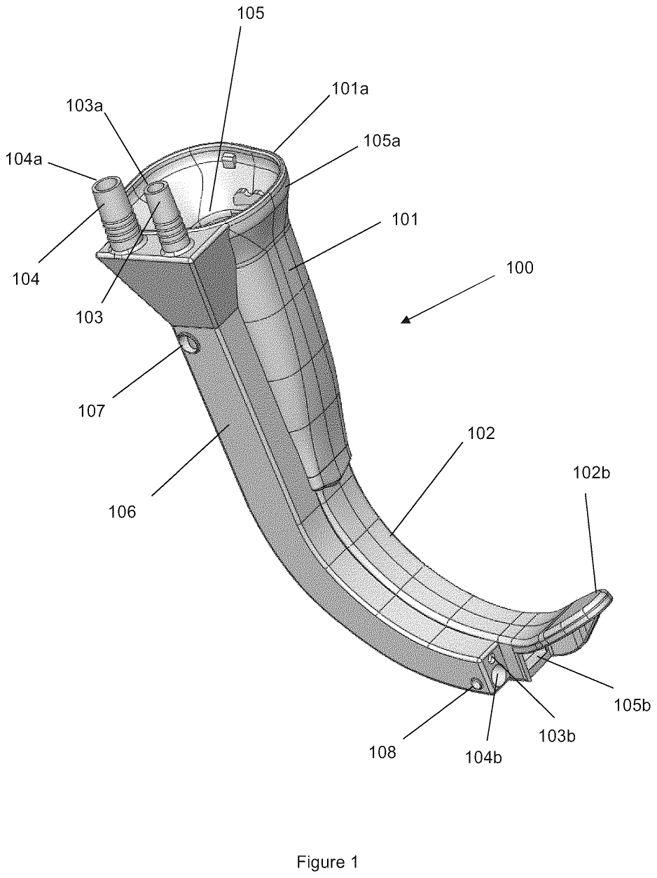

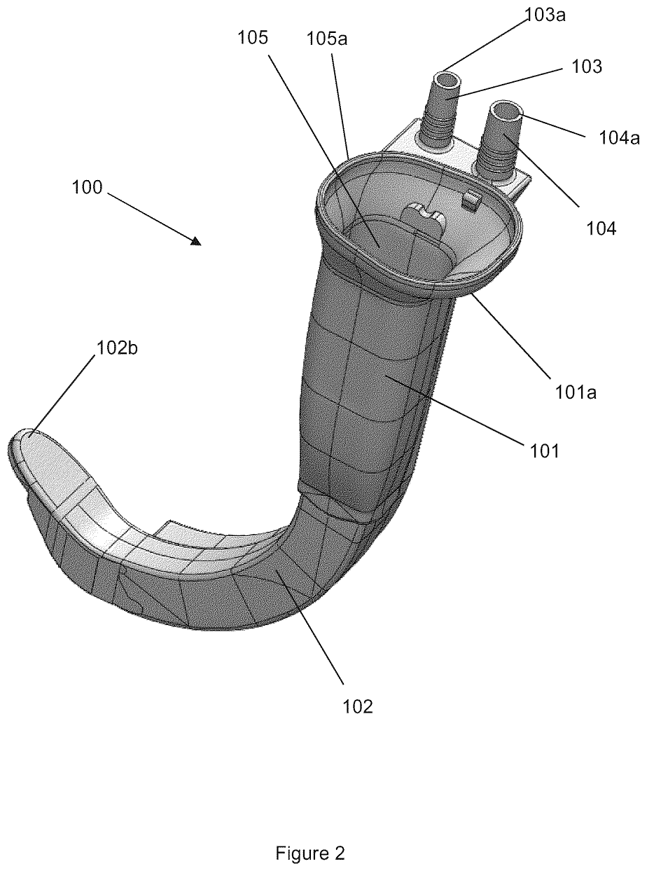

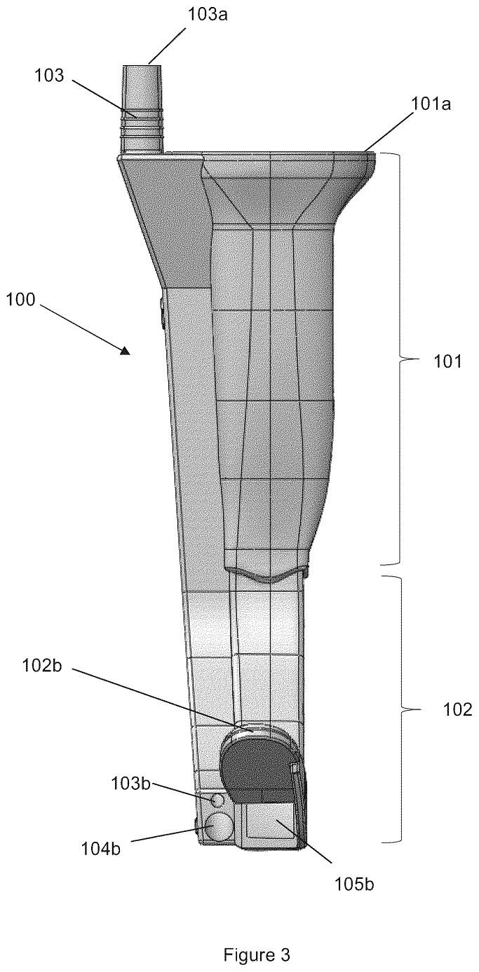

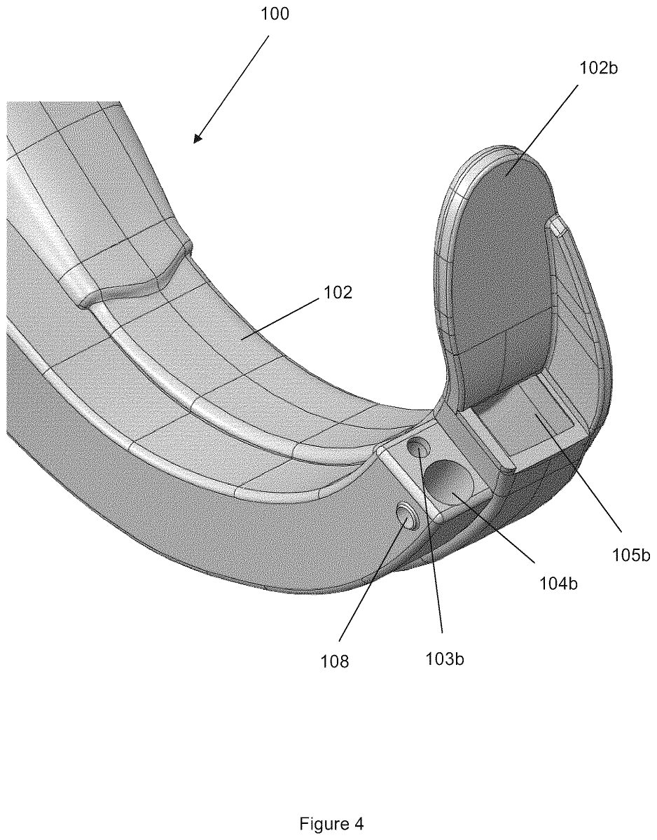

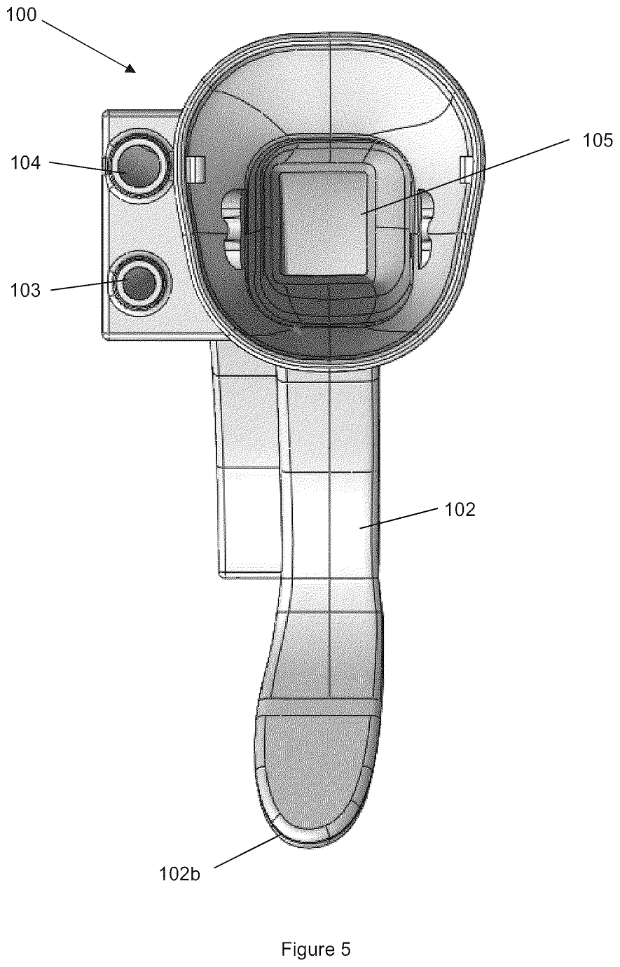

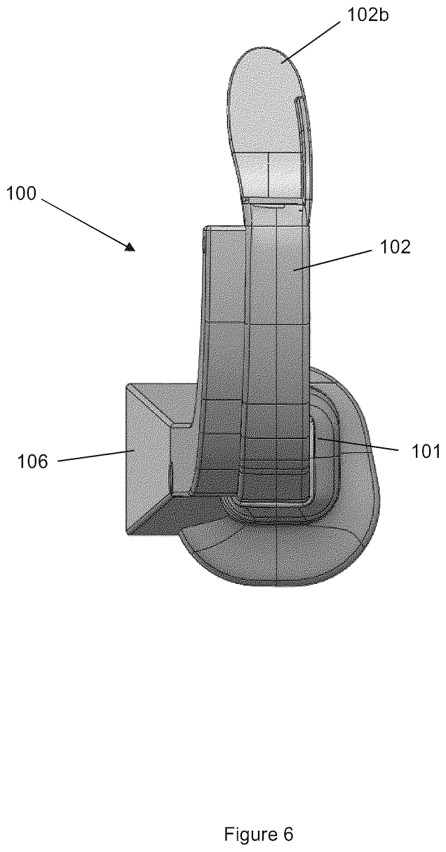

FIGS. 1 to 7 are schematics of an embodiment of the present laryngoscope device 100 for intubating a patient. The laryngoscope device comprises a handle element 101 at a proximal portion of the device coupled to a blade element 102 at a distal portion of the device. The device may be comprised of any suitable material that is capable of undergoing a sterilization process, including a metal alloy, such as those comprising titanium or stainless steel, or plastic. The device may be made of materials suitable for re-useable use or may be suitable for single-use only (i.e., disposable).

The handle element 101 is configured to be held in the hand of a user, preferably the left hand of the user.

The blade element 102 is configured for insertion into the airway of a patient. The blade element may be any suitable size and/or shape for use in intubating patients. In some embodiments, the blade element is straight. In other embodiments, such as the illustrated embodiment, the blade element is curved.

In some embodiments, the handle element 101 may be detachably connected to the blade element 102, such that a blade element can be exchanged with one or more other blade elements of differing sizes and shapes. In other embodiments, the blade element 102 cannot be detached from the handle element 101—e.g., the laryngoscope 100 is of a unitary construction, such as the illustrated embodiment.

The laryngoscope 100 further comprises a first lumen 103 and a second lumen 104. In some embodiments, such as that illustrated in FIGS. 1 to 7, the first and second lumen are disposed on a lateral side of the device. In other embodiments, the first and second lumen are disposed on a dorsal or ventral side of the device. In some embodiments, the first and second lumen are disposed on the same side of the device, adjacent to each other (such as in the illustrated example), whereas in other embodiments, the first and second lumen are disposed on different or opposing sides of the device. In some embodiments, the first and second lumen are enclosed in a housing 106 that is coupled or secured to the device (such as in the illustrated example).

In some embodiments, the first lumen 103 and second lumen 104 are coupled to at least a portion of the handle element 101 or a portion of the blade element 102. In some embodiments, the first and second lumen are coupled to just the handle element or just the blade element, whereas in other embodiments, such as the illustrated embodiment, the first and second lumen are coupled to both the handle element and blade element. In some embodiments, the first lumen 103 and second lumen 104 are coupled to only to a portion of the handle element 101, whereas in other embodiments, the first and second lumen are coupled to the entirety of the handle element. In some embodiments, the first lumen 103 and second lumen 104 are coupled to only to a portion of the blade element 102, whereas in other embodiments, the first and second lumen are coupled to the entirety of the blade element.

In some embodiments, the first lumen 103 and second lumen 104 are secured to at least a portion of the handle element 101 or a portion of the blade element 102. In some embodiments, the first and second lumen are secured to just the handle element or just the blade element, whereas in other embodiments, the first and second lumen are secured to both the handle element and blade element. In some embodiments, the first lumen 103 and second lumen 104 are secured to only to a portion of the handle element 101, whereas in other embodiments, the first and second lumen are secured to the entirety of the handle element. In some embodiments, the first lumen 103 and second lumen 104 are secured to only to a portion of the blade element 102, whereas in other embodiments, the first and second lumen are secured to the entirety of the blade element.

The first lumen 103 and second lumen 104 each comprise a first end, 103a, 104a and a second end, 103b, 104b. In some embodiments the first end 103a, 104a of each lumen is the same diameter as its second end, 103b, 104b, whereas in other embodiments, the first end 103a, 104a of each lumen is a different diameter than its second end, 103b, 104b. The first end of each lumen and second end of each lumen can be any suitable diameter, and are preferably between about 2 mm to about 10 mm in diameter, and more preferably between about 4 mm to about 8 mm in diameter. For example, in one embodiment, the inside diameter of the first end 103a of the first lumen is 5 mm, the inside diameter of the second end 103b of the first lumen is 3 mm, the inside diameter of the first end 104a of the second lumen is 5 mm, and the inside diameter of the second end 104b of the second lumen is 3 mm. In other embodiments, the inside diameter of the first end 104a of the second lumen is 6.5 mm and the inside diameter of the second end 104b of the second lumen is 6.2 mm.

In some embodiments, the first end of the first lumen 103a and the second lumen 104a are directly adjacent to a proximal end of the handle element 101a. In other embodiments, the first and second lumen extend beyond the proximal end of the handle element or do not extend all the way to the proximal end of the handle element, such that the first end of the first lumen 103a and second lumen 104a are not adjacent with the proximal end of the handle element 101a. In the illustrated embodiment, the first end of the first lumen 103a and second lumen 104a extend beyond the proximal end of the handle element 101a.

In some embodiments, the second end of the first lumen 103b and second lumen 104b are directly adjacent to a distal end of the blade element, or the tip of the laryngoscope, 102b. In other embodiments, the first and second lumen extend beyond the distal end of the blade element or do not extend all the way to the distal end of the blade element, such that the second end of the first lumen and second lumen are not adjacent with the distal end of the blade element—e.g., they are proximally offset relative to the distal end of the blade element. In the illustrated embodiment, the second end of the first lumen 103b and second lumen 104b do not extend to the distal end of the blade element 102b. More particularly, in this embodiment, the distal end of the blade element 102b extends past the second end of the first lumen 103b and second lumen 104b to form a tapered tip that is configured to hold the patient's tongue forward during the intubation procedure, as illustrated in FIGS. 1, 2, 4, 5, 6, and 7(b). While not wishing to be bound by any particular theory or mode of action, by holding the patient's tongue forward during the intubation procedure, the tongue can be held out of the way of the opening of the larynx during use of the device, which may assist in correct placement of the endotracheal tube during the procedure.

The first end of each lumen, 103a, 104a, comprises a connector element, the connector element being configured to reversibly connect to an external source. The connector element may be any suitable connecting means, including but not limited to a nipple and nut connector, a ribbed, universal connector, or a Christmas tree connector. In some embodiments, the connector element on the first lumen 103 is configured to reversibly (e.g., detachably) connect to a compressed gas source, such as oxygen. In some embodiments, the connector element on the second lumen 104 is configured to reversibly connect to a suction source, such as a vacuum pump.

The second end of each lumen, 103b, 104b, comprises a port element. The cross-sectional shape of the port element is not particularly restricted. Thus, the port element can comprising a cross-sectional shape which is rectangular, cylindrical, or circular. In some embodiments, the port element may comprise a filter element. This filter element may be useful to prevent gastric contents and foreign materials present in the airway from entering and blocking or clogging the lumen. The filter element can be any suitable filtering means, including mesh, fabric, a screen, a porous plate, a strainer, or grating. In some embodiments, the filter element is a grooved, rigid dome that extends outwardly from the lumen that permits the flow of fluid or gases through the grooves in the dome into or out of the lumen despite present of solid particles in the patient's airway when the device is in use.

In some embodiments, the first or second lumen may further comprise a valve element. Preferably, the valve element is interposed between the first end and second end of the first or second lumen and is configured to operate (e.g., move) between a first position to a second position. Preferably, when the valve element is in the first position, the first end and second end of the lumen are in fluid communication, or the passageway through the lumen is open. Preferably, when the valve element is in the second position, the first end and second end of the lumen are not in fluid communication, or the passageway through the lumen is closed. The position of the valve element can be controlled by an activation means that is configured to move the value element between the first and second positions.

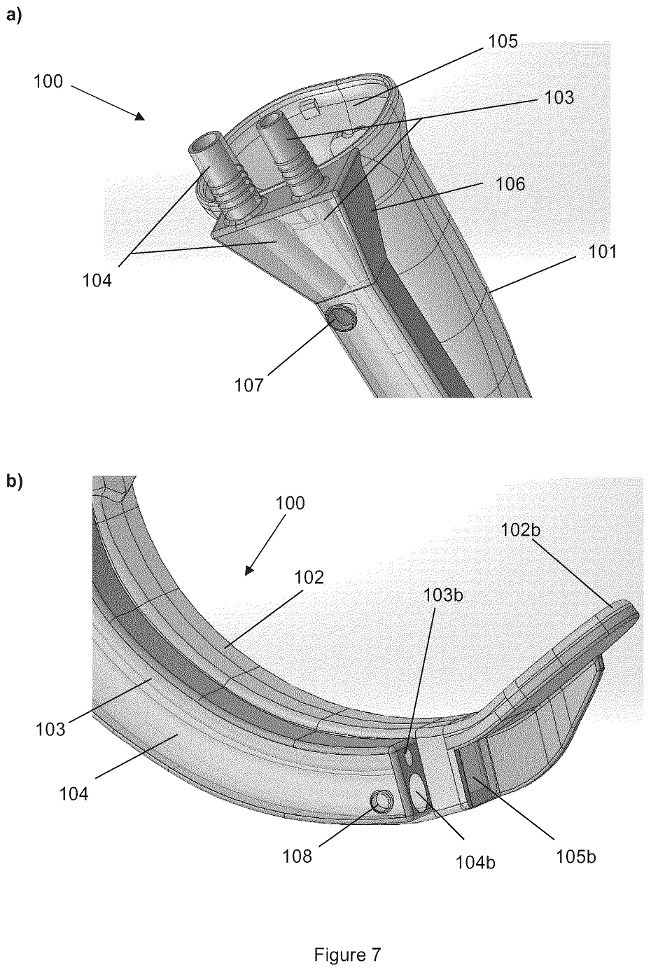

A preferred embodiment of a valve element is illustrated in the drawings. As illustrated in FIGS. 1 and 7, the value element comprises an aperture 107 in the lumen wall (such as in the illustrated example in FIGS. 1 and 7) and the activation means comprises a means of covering or blocking the aperture 107, such that when the aperture 107 is covered or blocked by the activation means, the value element is in the first position and when the aperture 107 is unblocked by the activation means, it is in the second position. By way of example, in such embodiments, the activation means can be a moveable cover or the user's hand. In such embodiments, the aperture can be any suitable diameter, and is preferably between about 2 mm to about 8 mm in diameter, and more preferably between about 3 mm to about 6 mm in diameter. In other embodiments, the activation means can include a button, trigger, switch, or lever. In some embodiments, the activation means comprises a spring. The activation means can be located on the device in a location that is easily accessible and useable by the user, such as on the handle element. This may be useful for users that want to control passage of air or liquid through the first or second lumen. For example, a user may want to control when and how much suction or oxygen is applied.

In some embodiments, one or both of the first and second lumen, 103, 104, comprise a second port element 108 proximal to its second end 103b, 104b. While not wishing to be bound by any particular theory or mode of action, the second port element may provide a secondary route for the flow of gases or suction into or out of the lumen if the second end of the lumen is blocked, such as by gastric contents or foreign materials. In the illustrated example, the second lumen 104 comprises a second port element 108.

The device may further comprise a third lumen with a first end and a second end. The third lumen can, for example, be used for delivery of medication or passing of instrumentation, such as a stylet. In some embodiments, the third lumen may be disposed on a lateral side of the device, in other embodiments, it may be disposed on a dorsal or ventral side of the device. In some embodiments, the third lumen may be disposed on the same side of the device as the first and/or second lumen, whereas in other embodiments, it may be disposed on different or opposing sides of the device as the first and second lumen.

The third lumen may be coupled or secured to at least a portion of the handle element or blade element. Like the first and second lumens, the third lumen may be coupled to just the handle element or just the blade element, or may be coupled to both the handle element and blade element. In some embodiments, the third lumen is coupled to only to a portion of the handle element, whereas in other embodiments, it is coupled to the entirety of the handle element. In some embodiments, the third lumen is coupled to only to a portion of the blade element 102, whereas in other embodiments, it is coupled to the entirety of the blade element.

Like the first and second lumens, the first end of the third lumen may be aligned with the proximal end of the handle element or may extend beyond it or not all the way to it. Similarly, the second end of the third lumen may be aligned with the distal end of the blade element or may extend beyond it or not all the way to it.

The first end of the third lumen may comprise a connector element, the connector element being configured to reversibly connect to an external source, such as a source of medication. The second end of the third lumen may comprises a port element, which may comprise a filter element.

The device may further comprise a chamber 105 contained within and traversing through the handle and blade elements of the device, as illustrated in FIG. 1. The chamber may be configured such that an image capturing device, such as a camera, can be inserted into the proximal end of the chamber 105a and positioned within the chamber. This may be useful for providing an indirect visualization of the patient's airway via a video feed while the patient is being intubated. The chamber can be any suitable diameter, and is preferably between about 2 mm to about 12 mm in diameter. The distal end of the chamber 105b may comprise glass or translucent plastic.

The present laryngoscope device 100 can be used in the following manner. The connector element on the first end 103a of the first lumen 103 is connected to a tube that is connected to an oxygen source, which is controlled at the source. In a preferred embodiment, the oxygen flow is set between about 0 L/min to about 15 L/min on an oxygen flowmeter. The connector element on the first end 104a of the second lumen 104 is connected to a tube that is connected to a suction source, which is controlled at the source. In a preferred embodiment, the suction level is set between about 0 to about 200 mm Hg. A camera that is connected to a video screen is inserted into the proximal end 105a of the chamber 105 and positioned within the chamber 105 such that the lens of the camera is adjacent to the distal end of the chamber 105b. The device 100 is then delivered to a patient during an intubation procedure like a regular laryngoscope, with the user holding the handle element 101 and inserting the blade element 102 into a patient's mouth, between the vocal cords, and into the trachea. The user may use the camera in chamber 105 to visualize the larynx on the video screen during the intubation process for correct placement of the device 100, while simultaneously providing suction and oxygenation using just one hand. In a preferred embodiment, such as that illustrated in FIGS. 1 and 7, the flow of suction through the second lumen 104 can be controlled by the user using the aperture 107 in the lumen wall, such that when the user's finger covers the aperture 107, there is suction at the second end 104b of the section lumen 104, whereas when the user does not cover the aperture 107, there is little to no suction at the second end 104b of the section lumen 104. In this way, the user can directly control the amount of suction being applied as the device 100 is being inserted and placed into the patient's trachea.

A cover for a laryngoscope is also described. This embodiment differs from that illustrated in FIGS. 1 to 7, in that the embodiment illustrated in FIGS. 1 to 7 is a laryngoscope device itself, whereas the cover can be used with a laryngoscope but is separate from the laryngoscope. The cover can be used for retrofitting a conventional laryngoscope with dual lumens as further described herein.

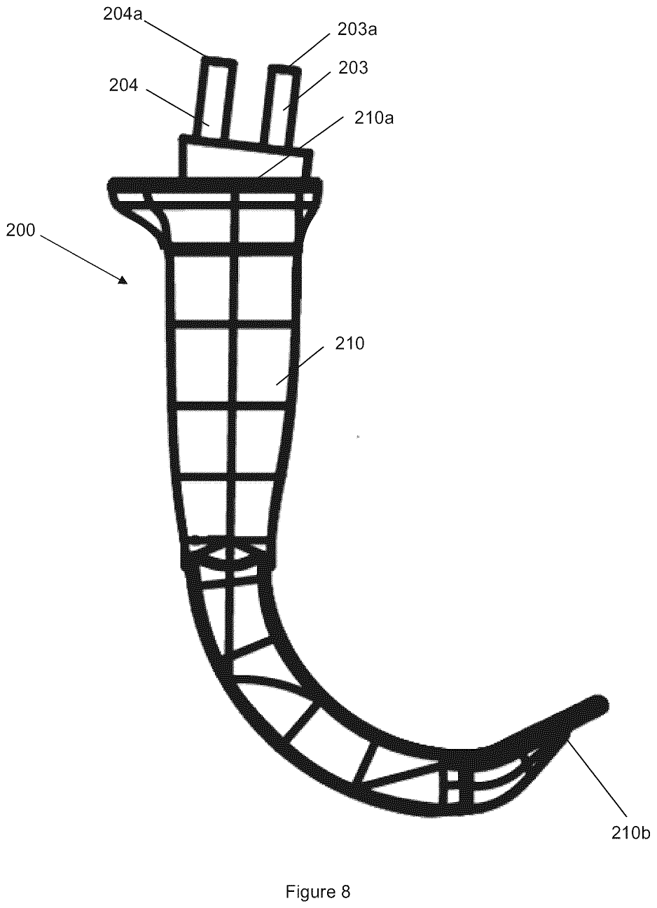

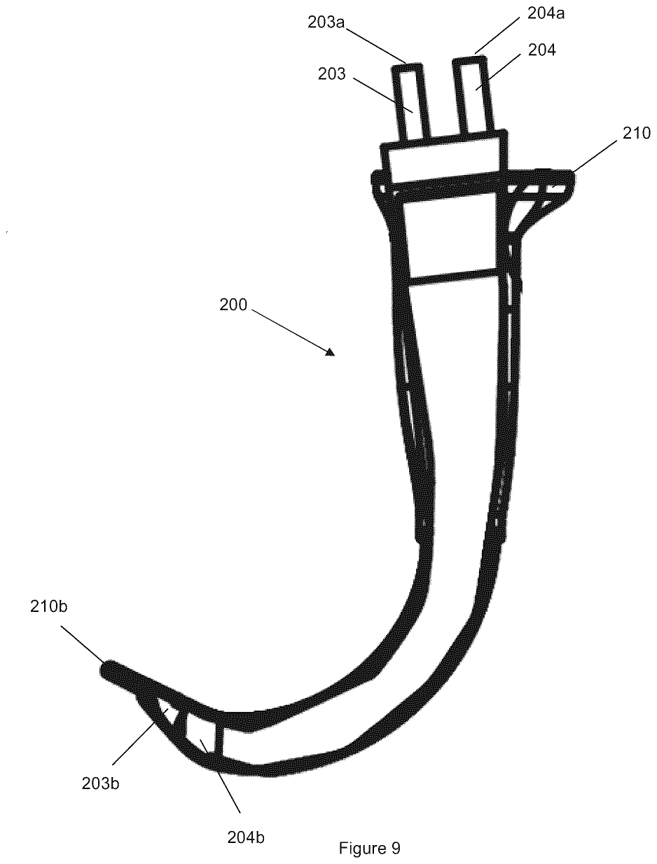

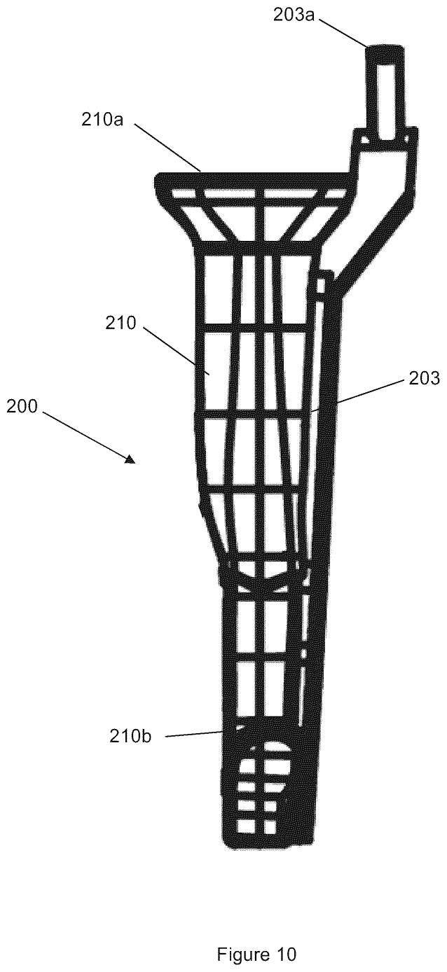

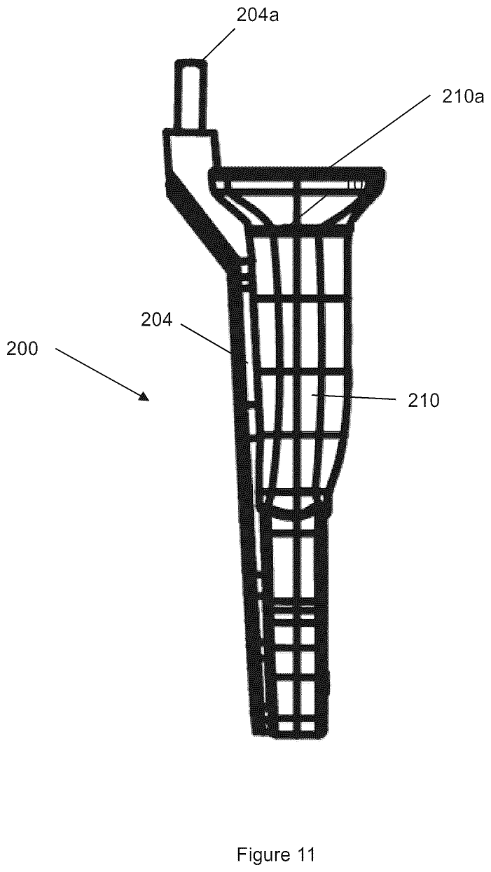

FIGS. 8 to 11 are schematics of an embodiment of the present laryngoscope cover 200 for intubating a patient.

The cover 200 comprises a sleeve 210 that has an open end 210a and a closed end 210b. The open end of the sleeve 210a is configured to receive the blade and handle of a laryngoscope, such that the sleeve 210 covers the blade and at least a portion of the handle. In some embodiments, the sleeve is configured to fit over the length of the blade until a distal end of the blade reaches the closed end of the sleeve 210b.

The cover further comprises a first lumen 203 and a second lumen 204 that are coupled to the sleeve 210. The first and second lumen may share similar or identical features as those described above for the first lumen 103 and second lumen 104 on the present laryngoscope device 100. For example, in some embodiments, the first and second lumen may be disposed a lateral side of the cover, whereas in other embodiments, they may be disposed on a dorsal or ventral side of the cover. In some embodiments, the first and second lumen may be disposed on the same side of the cover as each other, whereas in other embodiments, they may be disposed on different or opposing sides of the cover.

The first lumen and second lumen 203, 204 may comprise a connector element at a first end, 203a and 204a, the connector element being configured to reversibly connect to an external source, such as compressed gas or a suction source. The first lumen and second lumen, 203a and 204a, may comprise a port element at a second end, 203b, 204b, which may comprise a filter element. The first lumen and second lumen, 203a and 204a, may further comprise a valve element.

In some embodiments, the second end of the first lumen and second lumen is directly adjacent to the closed end of the cover. In other embodiments, the first and second lumen extend beyond the closed end of the cover or do not extend all the way to the closed end of the cover, such that the second end of the first lumen 203b and second lumen 204b are not adjacent with the closed end of the cover. In the illustrated embodiment, the second end of the first lumen 203b and second lumen 204b do not extend to the closed end of the cover 210b. More particularly, in this embodiment, the closed end of the cover 210b extends past the second end of the first lumen 203b and second lumen 204b to form a tip, which is configured to hold the patient's tongue forward during the intubation procedure.

The cover 200 may also further comprise a third lumen coupled to the sleeve, that may share similar or identical features as those described above for the third lumen on the present laryngoscope device 100.

For example, in some embodiments, the third lumen may be disposed a lateral side of the cover 200, whereas in other embodiments, it may be disposed on a dorsal or ventral side of the cover 200. In some embodiments, the third lumen may be disposed on the same side of the cover 200 as the first and second lumen 203, 204, whereas in other embodiments, it may be disposed on different or opposing sides of the cover as the first lumen 203 and/or second lumen 204.

The third lumen may comprise a connector element at a first end, the connector element being configured to reversibly connect to an external source, and a port element at a second end, which may comprise a filter element.

The cover 200 may be comprised of flexible material, such that it is configured to receive a laryngoscope of any shape or size, or the cover 200 may be comprised of rigid material, such that it is configured to receive one or more specific laryngoscopes, of a specific shape and/or size. The cover may be made of materials suitable for re-useable use or may be suitable for single-use only (i.e., disposable).

While not wishing to be bound by any particular theory or mode of action, the cover may be useful to users who wish to adapt an existing laryngoscope to enable simultaneous oxygenation and suction during use.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent, or patent application was specifically and individually indicated to be incorporated by reference in its entirety.

1. A laryngoscope device of unitary construction comprising:
(a) a handle element at a proximal portion of the device;
(b) a blade element at a distal portion of the device, the blade element configured for insertion into an airway of a patient; and
(c) a first lumen and a second lumen coupled to at least a portion of the handle element or the blade element.

2. The device of claim 1, wherein the first and second lumen are coupled to a portion of both the handle element and the blade element.

3. The device of claim 1, wherein the first and second lumen are coupled to the entirety of the handle element.

4. The device of claim 1, wherein the first and second lumen are coupled to the entirety of the blade element.

5. The device of claim 1, wherein the first and second lumen are coupled to the entirety of the handle element and blade element.

6. A laryngoscope device comprising:
(a) a handle element at a proximal portion of the device;
(b) a blade element at a distal portion of the device, the blade element configured for insertion into an airway of a patient; and
(c) a first lumen and a second lumen secured to at of a portion of the handle element or the blade element.

7.-8. (canceled)

9. The device of claim 6, wherein the first and second lumen are secured to the entirety of the blade element.

10. The device of claim 6, wherein the first and second lumen are secured to the entirety of the handle element and blade element.

11. The device of claim 1, wherein the first lumen and second lumen are disposed on a lateral side of the device.

12. The device of claim 11, wherein the first lumen and second lumen are disposed on the same lateral side of the device.

13. The device of claim 1, wherein the first and second lumen are disposed in a housing with a solid cross-section.

14. The device of claim 1, wherein each lumen comprises a first end adjacent to a portion of the handle element and a second end adjacent to a portion of the blade element.

15.-17. (canceled)

18. The device of claim 14, wherein the first end of each lumen comprises a connector element.

19.-22. (canceled)

23. The device of claim 14, wherein the second end of each lumen comprises a port element.

24. (canceled)

25. The device of claim 14, wherein one or both lumens comprise a valve element interposed between the lumen's first end and second end, the valve element configured to be reversibly movable from a first position to a second position, wherein when the valve element is in the first position, the first end and second end of the lumen are in fluid communication and when the valve element is in the second position, the first end and second end of the lumen are not in fluid communication.

26.-27. (canceled)

28. The device of claim 1, wherein the device comprises a third lumen coupled to at least one of a portion of the handle element and the blade element.

29. The device of claim 28, wherein the third lumen is coupled to a portion of both the handle element and the blade element.

30.-35. (canceled)

36. The device of claim 28, wherein the third lumen comprises a first end adjacent to a portion of the handle element and a second end adjacent to a portion of the blade element.

37. The device of claim 36, wherein the first end of the third lumen comprises a connector element.

38.-45. (canceled)

46. A cover for a laryngoscope, the laryngoscope comprising a handle element at a proximal portion of the laryngoscope and a blade element at a distal portion of the laryngoscope, the blade element being configured for insertion into an airway of a patient, the cover comprising:
(a) a sleeve comprising an open end and a closed end, the open end configured to reversibly receive the blade element and handle element of the laryngoscope, wherein the sleeve is configured to cover the blade element and at least a portion of the handle element; and
(b) a first lumen and a second lumen coupled to the sleeve.

47.-75. (canceled)
